(19) United States
(12) Patent Application Publication
(10) Pub. No.: US 2021/0330850 A1
(43) Pub. Date: Oct. 28, 2021

(54) Anti-Shadowing Ultraviolet "C" (UV-C) Virus Irradiation and Deactivation Chamber with Ozone Re-circulation and Neutralization

(71) Applicant: Roberto Catalan, Woodbridge, VA (US)

(72) Inventor: Roberto Catalan, Woodbridge, VA (US)

(21) Appl. No.: 16/859,088

(22) Filed: Apr. 27, 2020

(51) Int. Cl. A61L 9/12; A61L 9/20

(52) U.S. Cl. CPC A61L 9/122; A61L 2101/02; A61L 9/20

(57) ABSTRACT

An anti-shadowing virus irradiation and deactivation device comprised of air chambers having multiple ultraviolet “C” light sources, with a selectable recirculation path to harness ozone gas produced, and a system to neutralize said ozone gas upon exit.

### FEDERALLY SPONSORED RESEARCH

Not applicable

### SEQUENCE LISTING OR PROGRAM

Not applicable

### TECHNICAL FIELD

The embodiments of the present invention relates to the deactivation or neutralization of viruses (including bacteria and other harmful micro-organisms), specifically to a novel device for anti-shadowing, ozone recirculation and neutralization, primarily designed for hospital rooms where infected people are being treated, to significantly reduce risk to doctors and nurses on duty.

### BACKGROUND ART

The prior art in the field of neutralization of viruses and bacteria is commonly found in the large-scale ultraviolet treatment of drinking water and in food processing. These systems are typically situated in controlled industrial settings that are managed automatically and remotely. Examples of such prior art are found in U.S. Pat. No. 5,230,792 A by Sauska et al, U.S. Pat. No. 7,361,904 B2 by Cassauce et al, U.S. Pat. No. 6,565,803 by Calcon Carbon, U.S. Pat. No. 8,742,365 B2 by Abe, Kobayashi, et al, etc.

Other related prior art are in the field of HEPA air filter, bio-air purifiers and dust filtration devices. Examples of which are U.S. Pat. No. 6,464,760 by Sham, et al, U.S. Pat. No. 6,322,614 by Tillmans, U.S. Pat. No. 6,149,717 by Satyapal et al, etc.

Despite all the UV-C light-related prior art (as proof to the non-obviousness of the present invention), today doctors and nurses are treating infected patients while immersed in infected hospital room air, protected by nothing more than a layer of N95-rated face masks, surgical masks, and safety goggles or face shields. The tragic result thereof is that many doctors and nurses have been infected while on duty and subsequently added to the pandemic's roster of fatalities.

It is imperative therefore that we immediately find ways to go beyond passive prevention measures against COVID19 (e.g., physical distancing, wearing of masks in public, washing of hands, business lockdowns, prohibiting travel, etc.), and instead offer an active defense in a manner that is truly effective against the corona virus.

It is hoped that once the present invention is situated in every hospital room with COVID-19 patients, it will significantly reduce the risk of infection to doctors and nurses, and also contribute to a significant and much needed boost to morale.

Prior to this corona virus pandemic, hospital administrators in major cities around the world thought it was adequate to have only one or two negative pressure ‘bio-containment’ rooms. These special rooms were specifically designed for handling highly infectious patients harboring deadly diseases such as Ebola, MERS, bird flu, etc. However every bio-containment rooms needs to be embedded into the architectural design in the planning stage before a hospital is built. Each bio-containment room is also exceedingly expensive.

With the current COVID-19 pandemic, it is simply too cost prohibitive and unfeasible to rapidly retrofit all hospitals with these negative pressure rooms to accommodate the tens of thousands of infected people. The present invention would allow for the rapid creation of effective but inexpensive virtual ‘bio-containment’ rooms at nearly any location.

Likewise, this novel COVID-19 virus-neutralizing device is simple to manufacture, portable, and easy to operate.

### Objects and Advantages

### Several Objects and Advantages of the Present Invention are as Follows:

a) Unlike all prior art, the present invention uniquely addresses ‘shadowing’. Shadowing means that these nanometer-sized viruses can survive UV-C exposure if they are shielded (i.e. ‘shadowed’) by the floating particles to which they are attached. Any surviving COVID-19 virus can multiply with incredible speed once it has found a host. As such, the present invention has multiple UV-C light sources in one or more anti-shadowing chambers to consistently and repeatedly expose the viruses to the destructive wavelength of light from multiple angles.

b) Unlike all prior art, the present invention allows a very powerful quantity of harmful UV-C light and its equally harmful by-product ozone to be safely used inside a hospital room while patients, doctors, and nurses are present.

c) Unlike all prior art, the present invention harnesses the harmful ozone produced by UV-C light by re-circulating it through the system and uniquely includes the means to decompose the same ozone gas (O3) back to breathable oxygen (O2).

d) Unlike all prior art with a UV-C bulb that only shines briefly upon ambient or blown air, the present invention bombards infected air inside the array of anti-shadowing chambers with UV-C light and ozone gas repeatedly in a loop, for as long as deemed necessary to destroy the virus. The device has modes and cycles analogous to that of a washing machine, immersing the infected air inside the anti-shadowing chambers with UV-C and ozone in a similar manner to a ‘detergent’, until the air is ‘clean’ and sanitized.

e) Unlike UV-C used by prior art in the industrial water and food treatment, or the aforementioned bio-containment rooms in hospitals, the present invention is designed to be portable and relatively inexpensive, and can be used like an appliance in any room without extensive training or preparation.

All of the above features and benefits, taken together as a whole and implemented in the present invention, demonstrate its uniqueness and non-obviousness.

### Disclosure of Invention

The inventive subject matter disclosed herein describes a novel anti-shadowing ultraviolet C (“UV-C”) virus irradiation chamber with ozone recirculation and neutralization.

It cannot be stressed enough that the unique anti-shadowing feature of the present invention is an critical differentiating feature because the corona virus is so miniscule (only 0.06 to 0.12 microns) and so infectious (i.e. insanely rapidly multiplying) that all other air treatment devices without anti-shadowing—even if equipped with a UV-C light source would fail to adequately protect doctors and nurses who are attending to COVID-19-infected patients.

Ultraviolet “C” is the spectrum of light in the 100-280 nanometer range that is particularly destructive to viruses, bacteria and other harmful microbes. As a point of reference, sunlight that causes painful sunburns is only in the UV-“A” range. UV-“C” is far more damaging and dangerous to people. As such, UV-C light cannot be used in the open while people are present.

This novel, relatively portable device is specifically designed for virus deactivation and is uniquely differentiated because it allows dangerous quantities of UV-C light to be actively and safely used around people, such as in a hospital room with infected patients, doctors and nurses.

The present invention is unique in that it harnesses the by-product of UV-C light, which is ozone gas. Ozone is a powerful disinfectant against bacteria, pathogens and viruses, but is also very harmful. Humans can die if they breathe ozone gas in large quantities. Ozone is also very harmful to the environment. As such, the present invention creates ozone from the UV-C light and then re-circulates the ozone through the system to intensify its disinfecting capability. Once the virus is deemed destroyed, the present invention also neutralizes and decomposes the same ozone (O3) gas back into breathable oxygen (O2), so that the disinfected air that it expels is completely safe to breathe.

Anti-shadowing is accomplished via one or more spherical or rectangular chambers (other shapes can be obviously be used) which are equipped with multiple UV-C light sources. Typically, these light sources are generally UV-C bulbs if used in a spherical chamber, and long UV-C lamps if used in a rectangular chamber. Higher intensity light from mercury-type UV-C bulbs or lamps is recommended.

All internal parts of the device that are directly exposed to UV-C light are made of a material that is not affected by UV-C, such as acrylic and aluminium, or likewise covered, painted, or treated to counter any deterioration due to the harmful rays.

UV-C spectrum light emitting diodes (LEDs) exist in the market, but since LEDs do not produce much heat, the amount of ozone produced is negligible. However, the fact that hardly any ozone produced by UV-C LEDs is a bonus for smaller embodiments of the present invention and shall be discussed later.

Another important aspect of anti-shadowing is to process the viruses thoroughly in the UV-C bombardment chambers. One way that the present invention accomplishes this is by using small rotating louvers at the joints between the spherical or rectangular chambers. These louvers can be passively rotating (e.g. not powered) or actively controlled (e.g. using PCB or other very small motors) to ensure that the airflow inside the chambers are dispersed or swirled for a set amount of time, rather than allowing the air to form a single flowing mass into and out of each chamber. Such a well-formed air mass will protect viruses traveling at the middle of that air mass, so this feature is designed to prevent that.

Similarly, even if the UV-C light sources radiate intense rays, the virus can survive if the speed of the airflow is too high. We need to ensure that the virus is adequately exposed to UV-C light to ensure its destruction. As such the activation and speed of all fans used inside the device are synchronized or controlled separately via a microcontroller. Air speed of selected fans increase to allow more air in or out, and decreases or even stopping momentarily as required. The microcontroller allows the airflow to be customized for different kinds of rooms or areas in terms of size and purpose.

Air-flow optimized fans are used at the ingress and egress ports, while static pressure optimized fans are used inside the device. All of air that enters any of the ports and passageways of the device are one-directional. No air is allowed to return into the same port from which it came. This is accomplished using valves or flaps, controlled by servos, stepper motors or simple springs.

The device has five zones. The ‘hot’ zone is at and right after ingress where viruses are freely allowed to enter with no filters—since any filter will merely accumulate viruses. All parts of the device in the hot zone are designed to be removable to allow for easy cleaning and disinfection by a trained medical technician. It is also recommended that hot-zone parts be given a layer of medical-grade anti-bacterial silver or copper-infused paint, even if viruses may survive such treated surfaces.

The second zone is the ‘first pass’ zone. Infected air encounters its first UV-C light source in this pathway. This UV-C light is also in close proximity to the aperture used for recirculation of already processed air which has already been enriched with ozone gas.

While the recirculation cycle could be confined to the series of UV-C chambers alone (as an alternate embodiment), we are also using the higher levels of ozone gas accumulated with every cycle to ‘self-disinfect’ more surface areas inside the device that may contain live viruses.

The third zone is the ‘bombardment zone’, featuring one or more anti-shadowing chambers with multiple UV-C light sources. The arrangement and number of light sources is critical for successful anti-shadowing. It is recommended that the placement of UV-C light sources inside each spherical or rectangular chamber be different from any other chamber, in relation to each chamber's apertures. For example, in a spherical chamber, tetrahedral or octahedral arrangements for the UV-C bulbs should sufficiently address shadowing, but the same tetrahedral or octahedral arrangement should be different from the next spherical chamber in relation to the previous chamber's apertures. The varied UV-C light positions decrease the chance of any virus surviving the journey through all of the chambers.

With long UV-C lamps, the varied arrangement is slightly more challenging. However UV-C lamps will generally have stronger rays than UV-C bulbs.

The fourth zone is ‘ozone decomposition’. The aforementioned microcontroller-activated path for recirculation temporarily closes to allow the fully-processed air to enter a sub-system for decomposing ozone (O3) back into oxygen (O2). This is done with filters, covered by manganese oxide, nickel oxide, and activated carbon respectively. The chemical composition of these manganese oxide and nickel oxide pulls the extra oxygen molecule from ozone to convert it to oxygen. The activated carbon also assists in the process by capturing fresh-smelling but harmful ozone gas and neutralizing the odor in the process.

The fifth and final zone is the exit zone. Fully UV-C processed air is passed through a particle filter to capture any dislodged manganese oxide and nickel oxide particles, or any other airborne dust or dirt that may have traversed the system.

### BRIEF DESCRIPTION OF THE DRAWINGS

FIG. 1 An overall x-ray view of the device and its most visible parts

FIG. 2 An overall x-ray view of the device showing airflow direction with arrow marks

FIG. 3 Close-up view of a louver equipped circular joint for the spherical UV-C chambers

FIG. 4 Close-up view of three filters used for ozone decomposition and airflow-driving fans

FIG. 5 Shows typical use of the device, connected to the face mask of a COVID19 patient

FIG. 6 An overall x-ray view of a version of the device with rectangular UV-C lamp chamber

FIG. 7 An overall x-ray view of the device showing controller, power supply, cord, and plug

FIG. 8 A battery-powered, portable version of the device using light emitting diodes

### REFERENCE NUMERALS IN DRAWINGS

101 Ingress portal (infected air) and louvre

102 Spherical UV-C bombardment chambers

103 UV-C bulbs

104 Hose receptacles (infected air)

105 Ozone decomposition filters

106 Control Panel

107 Egress port and louvre

201 Ingress airflow-optimized fan

202 ‘Hot zone’ passageway

203 Non-return airflow flap

204 First UV-C light source

205 Recirculation aperture and one-way valve cover

206 ‘First pass zone’ passageway

207 Pressure-optimized fan (at entrance to UV-C chambers)

208 Second UV-C light source (at end of passageway)

209 Louver-equipped joints

210 L valve or V flap for recirculation path or exit path

211 Recirculation path

212 Exit path

213 Egress port particle filter and airflow-optimized fan as a single unit

301 Rotating louver on Spherical joint

302 PCB or other small electric motor

303 Spherical joint seals

401 Manganese oxide filter

402 Nickel oxide filter

403 Activated carbon filter

404 Inbound speed-controlled static pressure-optimized fans

405 Outbound speed-controlled static pressure-optimized fans

501 Patient breathing mask

502 Patient hose for exhaled infected air

503 Hose receptacle (one of five)

601 Rectangular UV-C bombardment chambers

602 UV-C lamps

603 ‘First pass zone’ pressure fan

604 Square version of louver-equipped chamber joints

701 Power supply

702 Microcontroller box

703 Power cable input port

704 Power cable and plug

801 Particle filter

802 Small fan

803 Anti-shadowing chamber

804 UV-C light emitting diodes

805 Lithium batteries

### DESCRIPTION OF EMBODIMENTS

For the sake of clarity, several parts such as internal wiring, sockets, connectors, particle filters at ingress and egress ports, UV-protective lining used inside the device, partitions, support structure for the chambers, roller wheels, handles, etc. have been omitted from the drawings.

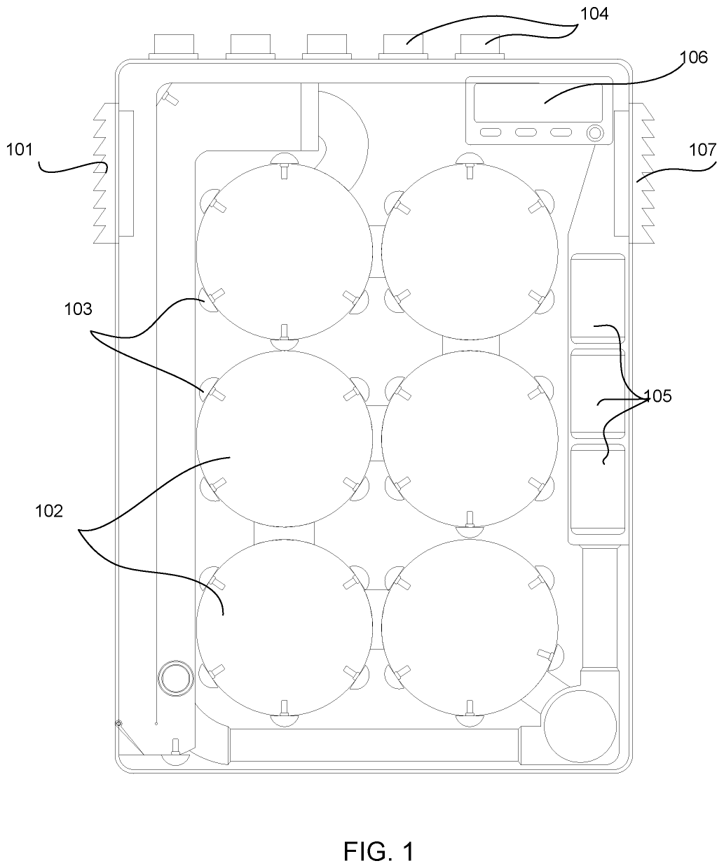

FIG. 1 is an overall x-ray view of the device and its most visible parts, beginning with the ingress portal (101) for infected air. This portal is protected by a louvre that matches the device pathways in design so that no UV-C light can be seen being emitted from its aperture. The louvre also prevents large objects or animals from entering into the device. There is no filter used at this portal because it would only cause viruses to aggregate at the filter rather than be processed by the device. A one-way flexible valve or flap (not shown) is attached to the ingress portal to prevent air from inside the device from exiting.

Also shown are the spherical anti-shadowing chambers (102) with multiple UV-C bulbs (103). These anti-shadowing chambers are where the viruses are repeatedly bombarded with UV-C to ensure its destruction. To the top of the device are hose receptacles (104) that gently draw in exhaled infected air from patients. Five hose receptacles are shown in this example, and each of these receptacles have one-way valves for flaps (not shown) to prevent air from inside the device to exit.

Any surface inside the device that is exposed to UV-C light should be lined, treated or painted with aluminium (not shown) or other UV-C resistant substance to prevent degradation.

Hoses attached to masks or tents on infected patients at one end, and to the present invention at the other end, is the most effective means for preventing doctors and nurses from being infected by COVID19 patients. Today, dangerous virus-laden air from infected patients are merely exhaled into hospital rooms and worse, unwittingly circulated throughout the hospital via the room's air conditioning vents, if that same room is not built with bio-containment.

Also shown are the ozone decomposition filters (105) which contain chemical elements that break down the ozone gas produced by the UV-C light sources back into oxygen (this will be discussed in greater detail later). The status of the device and various operating options is displayed and altered via a control panel (106). The device needs only to be powered on to provide its virus destroying abilities to a room.

The egress port (107) of the device has a louvre that likewise prevents large objects or animals from entering the device. The egress port also contains a particle filter (not shown). The air coming out of this egress port has been cleaned of viruses and ozone, and thus safe to breathe.

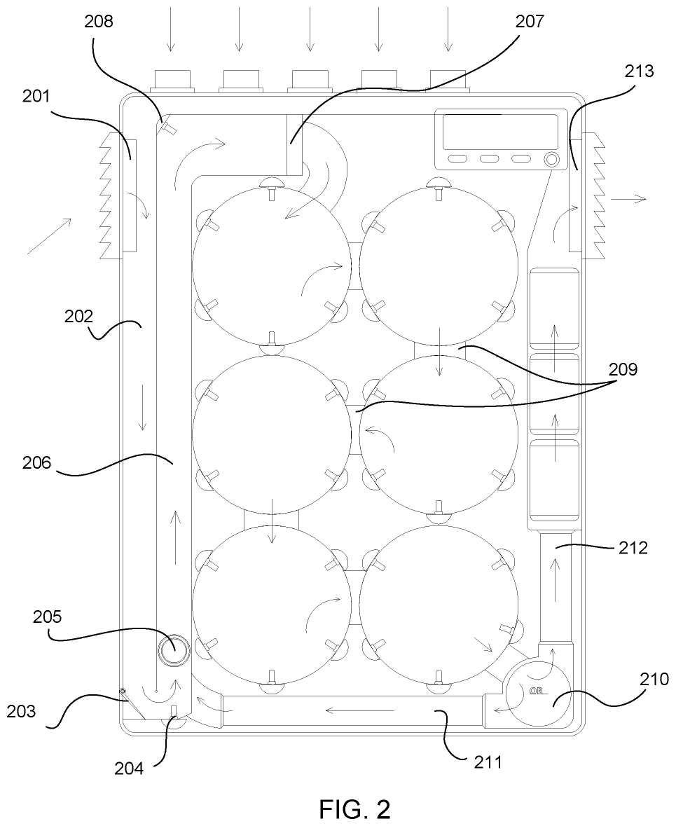

FIG. 2 shows an overall x-ray view of the device with arrow marks to depict how air travels through it. As mentioned previously the ingress port draws infected air through a one-way valve or flap (not shown). The negative air pressure is produced by an airflow-optimized fan (201) behind the ingress port.

The infected air then travels though the ‘hot zone’ passageway (202), meaning that the air here has not been processed in any way and still dangerous. This passageway serves as a buffer while the air further down the device is being processed to remove viruses. At the end of the ‘hot zone’ passageway is a non-return flap (203) that prevents infected air from going back up the passageway. Note that the non-return flap here can be replaced with an ‘L’ valve or other similar devices.

The first UV-C light source (204) then greets and partially disinfects the incoming virus-infected air from the ‘hot zone’. Note that LEDs, lamps, and other forms of UV-C light sources can be used here, not just bulbs. This first UV-C light source is at very close proximity to the recirculation aperture and one-way valve cover (205) which remains closed while the non-return flap of the hot zone is open. The partially disinfected air now travels up the first pass zone passageway (206) aided by a pressure-optimized fan (207) at the end of the first pass zone.

When the recirculation aperture is open and the hot zone flap is closed, processed air laden with ozone is drawn into the first pass zone passageway, driven by the aforementioned pressure-optimized fan. The ozone gas further disinfects the air and the passageway itself.

Towards the end of the first pass zone passageway is a second UV-C light source (208) that further assists in disinfecting the air and the immediate area near it. The aforementioned (speed-controlled) pressure optimized fan then forces the air into the entrance towards the spherical UV-C bombardment chambers.

Louver-equipped joints (209) at the entrance to the first spherical chamber and in between the rest of the spherical chambers ensures that the air inside the spheres are diffused or swirled so as to prevent the formation of a well-defined vortex of air running through one or more spheres, since such a well-defined airflow will protect viruses from being destroyed by the UV-C light.

A “L” valve or flap for recirculation path or exit path (210) is found at the end of the series of spherical chambers, the entrance to which can be aided by an optional static pressure-optimized fan (not shown). This L valve or flap is controlled by a microcontroller (not shown here) in unison with all of the fans and other valves or flaps.

Two airflow paths are available, one towards the recirculation path (211) and the other towards the exit path (212). At the end of the exit path is a particle filter (not shown) and an airflow-optimized fan (213) leading to the louvre-protected egress port.

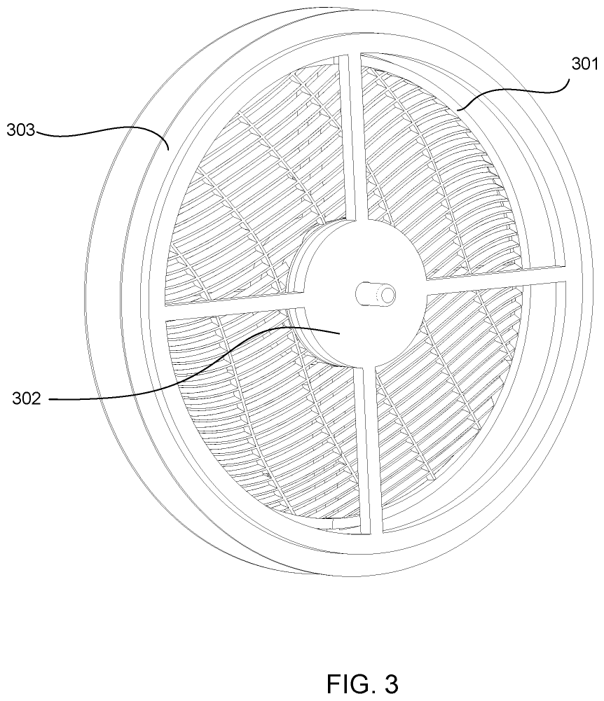

FIG. 3 shows a close-up view of the air diffusing louver-equipped circular joints (209) mentioned earlier, that is located in between the spherical anti-shadowing UV-C chambers. The rotating louvers (301) are either passive (non-powered) or moved by PCB motors or other small electric motors (302) which are connected to the microcontroller of the device (not shown here). Spherical joint seals (303) ensure that the infected air inside the spherical anti-shadowing chambers does not leak out of the system.

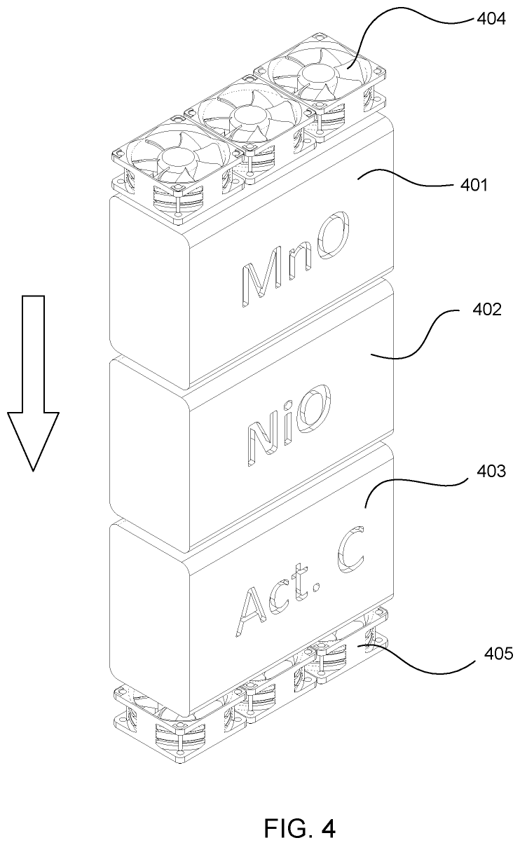

FIG. 4 is a close-up view of the three filters used for ozone decomposition. This is image is inverted in relation to the overall view in FIG. 2. The arrow symbol denotes direction of airflow. A set of static pressure-optimized fans (404) drive the now-disinfected air through the first filter in the airflow path, which is the manganese oxide filter (401), followed by the nickel oxide filter (402). Both chemical elements in these filters strip one molecule of oxygen from the harmful ozone (03) gas passing through it, to convert it into oxygen (O2).

Other methods and chemical compositions can be used to decompose ozone gas back to oxygen, and used instead of these filters if they are more efficient. An activated carbon filter (403) is used thereafter to remove the pleasant but harmful odor of ozone. Another set of speed-controlled static pressure-optimized fans (405) is placed at the end of this rather thick set of filters to push the now relatively ozone-free sanitized air out the egress portal of the device.

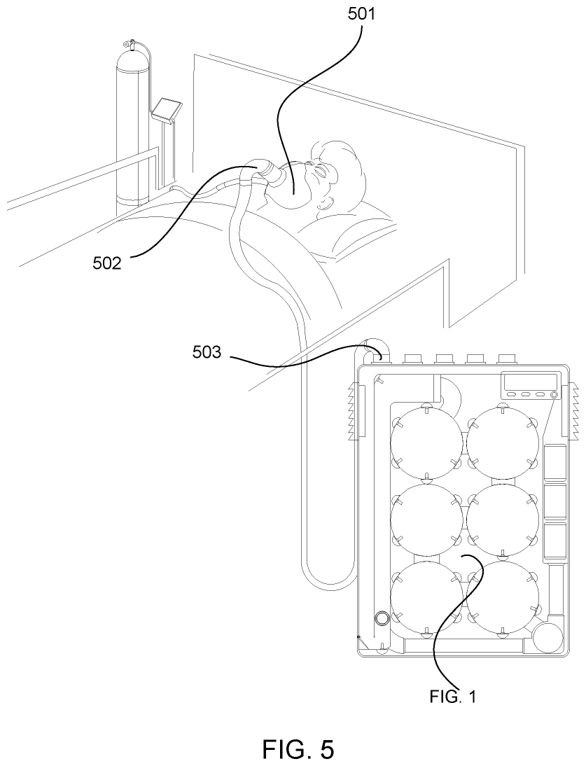

FIG. 5 shows a bed-ridden COVID 19 patient wearing a face mask (501). That face mask has an inhalation port that draws air from an oxygen-enriched air tank to the right of the patient. The face mask also has an exhalation port which is connected a hose (502) that is attached to the first of five hose receptacles (503) on the device shown in FIG. 1.

The use of the hose receptacles will significantly increase the number of effective ‘disinfected air changes per hour’ that the device can accomplish, and it can also reduce the physical size and cost of the device, depending upon the number of patients to be simultaneously serviced.

The most ideal setup in a hospital room is to use two separate devices, one for direct attachment to patient's masks or tents, and the other dedicated to processing the ambient air inside the same room. If used in this manner there will be nearly zero incidents of doctors and nurses getting infected by the patients that they are attending to.

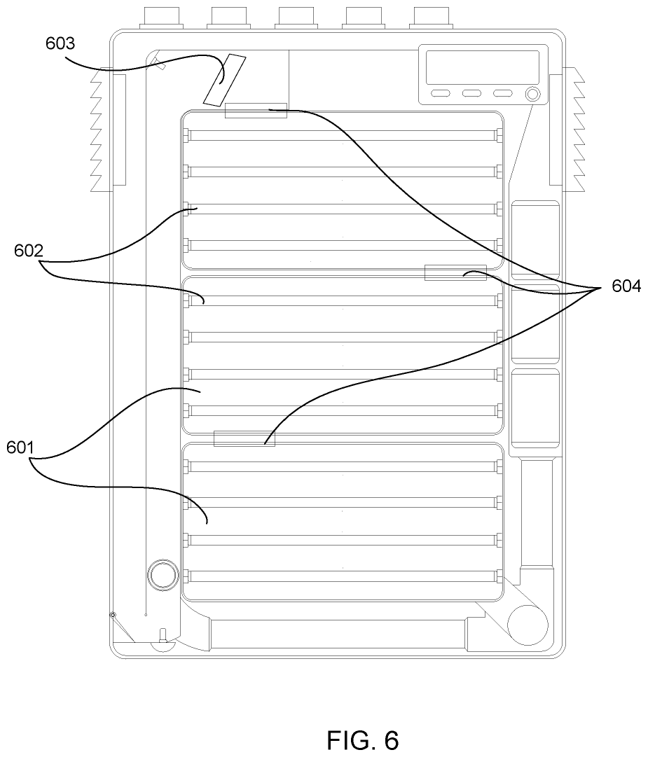

FIG. 6 shows an overall x-ray view of a different version of the device in FIG. 1 that instead has rectangular chamber (601) using long UV-C lamps (602). These chambers are easier to package, however the challenge as mentioned previously, is ensuring that the anti-shadowing capability is not compromised by equidistant spacing of the UV-C lamps. As such, variations in lamp locations (in relation to the prior chamber's apertures are recommended.

A static pressure-optimized fan (603) is used to drive the air into the first of the rectangular chambers. This fan is repositioned differently from that of the spherical chamber version. Louver-equipped chamber joints (604) this time with square-shaped seals are added at the entrance to and between these rectangular chambers. An optional static pressure optimized fan (not shown) can be added at the entrance of the previously mentioned “L” valve or flap to drive the air with more force towards the recirculation or exit paths.

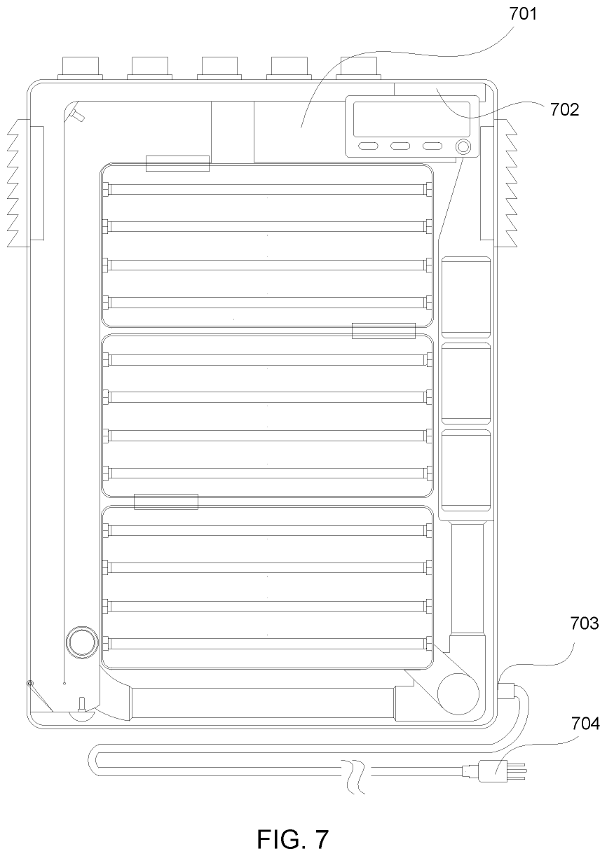

FIG. 7 is another overall x-ray view of the device, this time showing the location of the power supply (701), the microcontroller box (702), the power input port (703) and the attached cord and power plug (704).

Various sensors (not shown) can be added to the device to increase safety. These sensors can warn doctors and nurses of any anomaly (e.g. fan failure, bulb burned out, airflow is blocked, internal temperature is too high, etc.) via a buzzer and warning message on the control panel (106).

The fans, servos, motors, microcontroller, power supply, optional sensors and other electronic parts such as connectors, wires, and crimping tools can be bought from Adafruit.com, Sparkfun.com, Polulu.com, Digikey.com, Mouser.com, HobbyKing.com, MicroCenter.com and Amazon.com.

Acrylic pipes, aluminium sheets, enclosures, spherical and rectangular chambers, valves and flaps can be bought from ePlastics.com, U.S. Plastics.com, Plastic-Domes-Spheres.com, Home Depot, Lowe's, and Amazon.com.

The UV-C light sources in bulb, long lamp, and LED forms and their matching sockets, bases and reflectors can be bought from AtlantaLightBulbs.com, IntlLightTech.com, 1000 Bulbs.com, Mouser.com, Digikey.com, Boston Electronics (BosElec.com), Amazon.com, Home Depot and Lowe's.

Filters and activated carbon can be bought from Home Depot, Lowe's, and Amazon.com. The manganese oxide, nickel oxide can be bought from Amazon.com, AmericanElements.com and SigmaAldrich.com. Medical grade hoses and receptacles can be bought from WTFarley.com, PrecisionMedical.com and OhioMedical.com.

It is recommended that if any parts used in the device are 3D printed, they must be covered with metallic paint to protect against UV-C light. 3D filaments are not affected by UV-C light can be used, but these are generally expensive. 3D printing filaments, parts and supplies can be sourced from Ultimaker.com, E3D-online, MatterHackers.com, and Amazon.com. 3D printers can be sourced from Prusa Research, Lulzbot, Voron Designs, and many others.

The overall combination of the features of the present invention is unique and non-obvious. Further evidence is the fact that hospitals and clinics currently attending to COVID-19-infected patients are not using any similar anti-shadowing virus-destroying device.

### Description of Alternate Embodiments

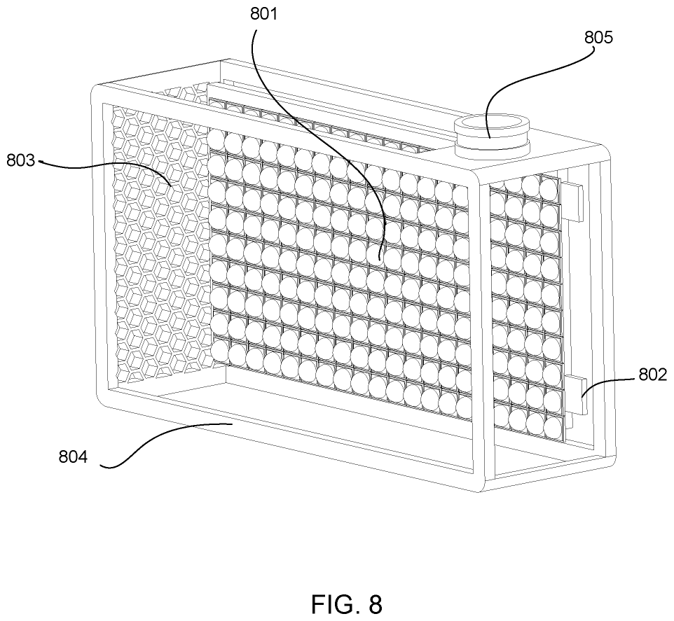

FIG. 8 shows an open frame of a portable anti-shadowing UV-C virus irradiation chamber and deactivation device for personal use, which can fit on a belt. The sidings of this portable device have been removed for clarity. An array of UV-C light emitting diodes (801) is shown. Behind these LEDs is a lithium battery (802) in pouch form that is roughly the same size as the UV-C LED array. The positive and negative terminals of the battery are clearly seen. The microcontroller (not shown) of the device is positioned behind the battery for protection.

Opposite to the array of UV-C LEDs is another identical array (not shown). The frame of the device (804) has a grill with a miniaturized louvre design (805) and a HEPA filter (not shown) to minimize UV-C light from being seen from the outside. As air enters the grill of the portable device, it is immediately bombarded by UV-C light from both facing LED arrays. Air is drawn by small fans (not shown) into another particulate filter (not shown), towards the hose receptacle (805) to which a breathing hose (not shown) is attached. This breathing hose leads to the intake port of a face mask (not shown, but similar to item number 501 in FIG. 5) for doctors and nurses.

The same parts shown in FIG. 8 can be modified and embedded into the back side of a full face mask so as to remove the need for a separate breathing hose. This miniaturized version of the present invention is yet another level of active protection for doctors and nurses who are attending to COVID19 infected patients. It will certainly provide far more protection than a passive N95 mask.

Face masks (3M brand is recommended), including snorkeling or diver-type full face masks can be bought from the following stores: Amazon.com, Home Depot, Lowe's, Scuba.com, Walmart.com, ScubaCenter.com, PADI.com, and AmericanDivingSupply.com. Modified parts for these face masks are normally 3D printed, but must be covered in paint or other safe filler material since the printed part can have air holes between layers if printed incorrectly.

### Operation—Best Mode for Carrying Out the Invention

The device is simple to operate. Once plugged into a wall socket and positioned in the hospital room it needs only to be powered on.

It would be best to position the device in the center of the room if possible. An optional hose attachment (mentioned earlier) allows face masks or intubation tubes of infected patients to be directly attached to the device. Doing so would immediately make the room significantly safer for attending doctors and nurses.

The present invention has two operating modes: ‘recirculation mode’ and ‘simple pass-through’ mode. Recirculation mode is the default.

In the ‘recirculation mode’, the device fills the pathways and all anti-shadowing chambers with air from the outside. The collected air is bombarded with UV-C light and then re-circulated back into the system to harness the ozone gas that was generated by the UV-C light sources. This re-circulation process is repeated as necessary.

It must be emphasized that the objective of the recirculation measure is to increase the safety margin that the processed air has been fully disinfected of viruses.

Thereafter, the now-disinfected but ozone-laden air is forced through a series of filters, which decomposes ozone (O3) back into oxygen (O2) before the disinfected air is released through a particulate filter back into the room.

In contrast, the ‘simple pass through mode’ does not avail of recirculation. Instead, the air travels through the system linearly, with the device's fans generally rotating at a slower velocity. The UV-C light sources are run at their maximum illumination and voltage to compensate for the loss of exposure time gained if recirculation was enabled.

However, simple pass through mode is more appropriate for UV-C lamp with higher ‘lumens’, to match the same ‘safety factor’ provided by recirculation mode.

It is recommended that the use of the present invention be limited to hospitals and medical clinics. It is not intended for casual household use, except for safely isolating an infected family member. UV-C and ozone are not selective and can also destroy other micro-organisms that are beneficial or even essential to human life and all other forms of life.

### Conclusions, Ramifications and Scope

The lethality and incredible infectiousness of COVID-19 caught the entire world by surprise. To date two million people have been infected, up from one million people just weeks earlier. Around the world, and tens of thousands have died.

The norms of daily life across the globe changed. Physical distance was imposed, travel is restricted, meetings in groups banned. People are now too scared of getting infected such that normal, formerly genial social interactions have started to fray.

We cannot afford to continually lose doctors and nurses to COVID-19 or other pandemics, because if we do, that would cause irreparable panic and a collapse of civility, if not civilization.

The present invention enables virus-destruction capability despite close proximity to people. That unprecedented capability is vital to restoring the confidence of the general public, and will greatly assist in bringing daily life back to a semblance of normality.

Worrisomely, several medical studies from different countries have reported that some COVID19 patients who have supposedly recovered from the virus have been found to be re-infected.

Likewise, some medical experts have voice their concern that COVID19 may become a seasonal disease, similar to the flu, except that COVID19 is much more lethal. The potential for such a permanent threat makes the versatility and active protection provided by the present invention simply invaluable.

Because of the urgent need to prevent more loss of life among our heroic doctors, nurses, and infected patients, I would like to request expedited processing of this application, including the permission to discuss it openly—immediately, so that I can start to gather the needed resources to manufacture and distribute this device to hospitals and medical clinics everywhere.

### INDUSTRIAL APPLICABILITY

The industrial applicability of this shielded anti-shadowing ultra violet “C” virus irradiation and deactivation chamber with ozone re-circulation and neutralization should be self-evident, most especially since the COVID-19 virus epidemic is still raging globally today.

I claim:

1. A virus irradiation and deactivation device comprised of, a plurality of fans, a uni-directional passageway, one or more chambers equipped with multiple UV-C light sources, and a means to decompose ozone (O3) gas into oxygen (O2).

2. The device of claim 1, further comprised of an airflow recirculation passage, airflow control valves, and a microcontroller and software to coordinate all functions.

3. The device of claim 2, further comprised of rotating louvers to disperse air inside said chambers.

4. The device of claim 3, further comprised of receptacles to attach hoses from infected patient's masks or tents,

5. The virus irradiation and deactivation device of claim 11 further comprising wherein said UV-C light sources are light emitting diodes.

6. The device of claim 5, further comprised of, batteries, and an attachment to an intake port of a face mask.

7. A method for disinfecting and/or decontaminating air contaminated by airborne viruses safely in the presence of people in an environment, comprising:
(a) drawing said air using one or more fans through a uni-directional airflow passageway,
(b) directing said air from said airflow passageway into one or more chambers, wherein each of said one or more chambers is equipped with multiple UV C light sources, and
(c) decomposing ozone produced by said UV-C light sources back into oxygen, and
(d) discharging decontaminated air from step (c) into said environment.

8. A virus irradiation and deactivation device comprising,
an opaque enclosure, said enclosure having an air ingress port and air egress port, said air ingress port connected to a hot zone conduit,
a first-pass conduit, said first-pass conduit joined at a junction to said hot zone conduit wherein a non-return airflow flap is disposed at said junction, said first-pass conduit having a recirculation aperture,
a first UV-C source, said first UV-C source positioned in said first-pass conduit,
said first pass conduit connected to a single bombardment chamber or a first bombardment chamber of a plurality of bombardment chambers which are connected in series, said plurality of bombardment chambers having a first bombardment chamber and a last bombardment chamber, said first bombardment chamber having a plurality of UV-C sources,
a pressure optimized fan positioned to draw air sequentially into said first-pass conduit, into said single bombardment chamber or said plurality of bombardment chambers, and to a bifurcation valve or flap, said bifurcation flap connected either to said single bombardment chamber or to the last bombardment chamber in the plurality of bombardment chambers, said bifurcation valve or flap configured to direct airflow to either a recirculation conduit or to an exit conduit, said exit conduit terminating at said egress port,
said recirculation conduit further connected to said recirculation aperture wherein air directed into said recirculation conduit from said bifurcation valve or flap re-enters said first-pass conduit,
means for controlling fan speed and power to the UV-C sources, coordinating opening and closing of the valves and/or flaps,
means for providing electrical power to the device.

9. The virus irradiation and deactivation device of claim 8 further comprising wherein said single bombardment chamber or said plurality of bombardment chambers are spherical, and further comprising wherein each of the spherical bombardment chambers has UV-C sources arranged in either:
(a) a tetrahedral arrangement, or
(b) an octahedral arrangement.

10. The virus irradiation and deactivation device of claim 9 further comprised of rotating louvers arranged to disperse air inside each of the spherical bombardment chambers.

11. The virus irradiation and deactivation device of claim 3 further comprising wherein said UV-C sources are arranged in either:
(a) a tetrahedral arrangement, or
(b) an octahedral arrangement.
